(19) United States
(12) Patent Application Publication
(10) Pub. No.: US 2022/0227972 A1
(43) Pub. Date: Jul. 21, 2022

(54) NATURAL COMPOSITION COMPRISING ALGINATE AND CELLULOSE NANOFIBERS ORIGINATING FROM BROWN SEAWEED

(71) Applicants: Linn BERGLUND, Luleå (SE); Kristiina OKSMAN, Luleå (SE)

(72) Inventors: Linn BERGLUND, Luleå (SE); Kristiina OKSMAN, Luleå (SE)

(21) Appl. No.: 17/615,404

(22) PCT Filed: Jun. 5, 2020
(86) PCT No.: PCT/EP2020/065585

§ 371 Date: Nov. 30, 2021

(30) Foreign Application Priority Data
Jun. 5, 2019 (SE) 1950674-0

(51) Int. Cl. C08L 1/04; C08B 15/02; C08J 3/24; C08B 15/10; A61L 27/52; A61L 27/20; A61L 27/26; B33Y 70/00; B33Y 40/10; B29C 64/314

(52) U.S. Cl. CPC C08L 1/04; B29K 2401/00; C08J 3/24; C08B 15/10; A61L 27/52; A61L 27/20; A61L 27/26; B33Y 70/00; B33Y 40/10; B29C 64/314; C08J 2305/04; C08J 2401/02; C08L 2203/02; C08L 2312/00; A61L 2430/34; C08B 15/02

(57) ABSTRACT

A natural composition for 3D printing comprising alginate from brown seaweed and cellulose nanofibers, wherein the cellulose nanofibers originate from cellulose from the same brown seaweed sample(s) as the alginate.

### TECHNICAL FIELD

The present document relates to a natural composition and shaped material comprising alginate from brown seaweed and cellulose nanofibers originating from cellulose from the same brown seaweed sample(s) as the alginate, to a use of such a natural composition and to methods for production of such a natural composition and shaped material.

### BACKGROUND ART

Brown seaweed is a promising natural resource for carbohydrate extracts. The polysaccharides in brown seaweed differ profoundly from those found in terrestrial plants. Though cellulose is present in smaller fractions, alginate is the major structural component of the cell wall. Thus, the most common source of alginate for commercial uses is from brown seaweed. (Misurcova et al., 2012)

Alginate, consists of 1,4-glycosidically linked α-L guluronic acid (G) and β-D-mannuronic acid (M). The linear chains are made up of different blocks of guluronic and mannuronic acids referred to as MM blocks or GG blocks (MG or GM blocks), where the linkage in block structure results in varying degrees of flexibility or stiffness in alginates. In the presence of Ca²⁺ the GG blocks form ionic complexes to generate a stacked (cross-linked) structure known as the “egg-box model”responsible for the strong gel formation (Peteiro et al 2018).

This behaviour of alginate has been widely utilized in the assembly of hydrogels for biomedical applications such as cartilage- (Markstedt et al., 2015; Naseri et al., 2016) and bone- (Abouzeid et al., 2018) tissue engineering. 3D printing of alginate have triggered increased attention in the assembly of hydrogels for biomedical purpose, where a main challenge lies in achieving shape fidelity of the 3D structure. Although the viscosity of alginate can be adjusted through its concentration and molecular weight (Kong et al., 2002), its rheological behaviour is not sufficient for structural integrity while printing. Several researchers have solved this by introducing cellulose nanofibers (CNF) from e.g. wood or wood pulp to engineer the alginate as ink, suitable for 3D printing (Chinga-Carrasco, 2018 and WO2016/128620 A1), where the direct cross-linking ability of alginate with the shear thinning behaviour of CNF can be combined.

CNFs are further attractive for biomedical applications owing to their good mechanical properties and biocompatibility. The introduction of CNFs has shown very promising results, where an increased viscosity combined with shear-thinning behaviour have enabled printing of complex 3D shapes (Markstedt et al., 2015). In addition, a reinforcing effect of CNF by a significant increase in compressive properties have been reported (Abouzeid et al., 2018). In a recent study, CNFs has not only shown to be beneficial for dimension stability and mechanical properties, the presence of an entangled nanofiber network has further shown to affect the pore structures, enhancing its size, thus making it more suitable for cell growth (Siqueira et al., 2019).

Both alginate and CNFs can be isolated from renewable sources, though often associated with relatively energy intense and extensive processing steps (McHugh, 2003; Falsini et al., 2018). Hence, it would be desirable to provide an alginate/CNF ink suitable for 3D printing, where the preparation process is less extensive and energy intense and more resource efficient than known processes.

### SUMMARY OF THE INVENTION

It is an object of the present disclosure to provide an alginate/CNF ink and a method of preparing such an ink, which method is less extensive and energy intense and more resource efficient than known processes. Further objects are to provide a method for providing a shaped material from such alginate/CNF ink, a use thereof for providing a shaped material and to provide the shaped material as such.

The invention is defined by the appended independent patent claims. Non-limiting embodiments emerge from the dependent patent claims, the appended drawings and the following description.

According to a first aspect there is provided a natural composition for 3D printing comprising alginate from brown seaweed, and cellulose nanofibers, wherein the cellulose nanofibers originate from cellulose from the same brown seaweed sample(s) as the alginate.

Such a natural composition or biogel may be suitable as ink for 3D-printing. As the alginate and the cellulose are from the same resource, from the same sample(s) of brown seaweed (Phaeophyceae), such a natural composition is more resource efficient than known inks wherein aliginate may be extracted from e.g. a brown seaweed and cellulose extracted from wood or wood pulp.

The natural composition has a composition of alginate and cellulose as found in the brown seaweed sample(s), i.e. the content of cellulose and alginate and the ratio of cellulose to alginate in the natural composition is the original content and ratio of the cellulose and alginate in the brown seaweed sample(s). With biogel is here meant a gel comprising one or more components (cellulose and alginate) that are natural or recombinant biological components.

A solid content of the natural composition may be 2-10 wt %. In one example the solid content is 2-5 wt %.

According to a second aspect there is provided a method for preparing a natural composition comprising alginate and cellulose nanofibers, wherein the method comprises the steps of providing a material of brown seaweed, purifying the material to remove impurities from the brown seaweed comprising the alginate and cellulose, and nanofibrillating the cellulose of the purified material.

The material of brown seaweed may consist of or comprise the whole seaweed plant, i.e. the holdfast (root-like), the stipe (stem-like) and the blade (leaf-like) structure, or alternatively, only one or two of these parts. The material may e.g. be fresh seaweed, seawed which has been put in the freezer and thawed before use, or sundried seaweed soaked before use.

Before the purification step, the material may be cut into smaller pieces. Purification is performed to remove colour pigments and other impurities from the material. After purification there is a step of washing the material, for example in water, to remove bleaching chemicals. Washing should be performed until a neutral pH is reached.

The nanofibrillation method used may be any nanocellulose fibrillation known in the art. Nanofibrillation of the material may for example take place using a supermasscolloider ultrafine friction grinder.

With this method the cellulose and the alginate are from the same brown seaweed sample(s). The present method is, hence, more resource efficient than known processes and less extensive as alginate and cellulose are processed simultaneously from the brown seaweed sample(s). It was shown that with the present method measured energy consumption for the nanofibrillation step was lower than energy consumption for nanofibrillation of commercial wood kraft pulp, less than 1.5 kWh/kg compared to about 8.4 kWh/kg under similar processing conditions (Berglund et al., 2017). The low energy demand suggest that the presence of alginate during the nanofibrillation step may be beneficial for the separation of nanofibers. The method is, hence, less energy intense than production processes involving alginate from one source and cellulose nanofibers originating from another source, where the cellulose is nanofibrillated prior to being mixed with the alginate.

The step of purifying the material may comprise the use of one or more cellulose bleaching substances.

The one or more cellulose bleaching substances may be conventional cellulose bleaching substances or chemicals used in pulp production. In one example NaClO₂ in an acetate buffer may be used.

According to a third aspect there is provided a method for preparing a shaped material, a matrix, comprising alginate and cellulose nanofibers, the method comprising the method described above, and further steps of: forming a shaped material of the natural composition, and crosslinking the alginate. The crosslinked shaped material forming a hydrogel.

After nanofibrillation, the natural composition may be formed into a shaped material, for example by 3D printing. The step of crosslinking the alginate of the composition may take place by crosslinking the shaped material by for example adding a crosslinking agent to the shaped material. The shaped material may for example be soaked in a crosslinking bath. Alternatively, crosslinking may take place as the shaped material is formed. In yet an alternative, crosslinking may take place by adding a crosslinking agent to the composition before forming the shaped material. Suitable alginate crosslinking methods and agents are well-known in the art.

The cross-linking degree of alginate may vary depending on the cross-linking method used, the type of shaped material, and the required properties of the shaped material.

The step of crosslinking the alginate may comprise the use of a bivalent or trivalent cation, a peroxide, a vinylsilane, UV light, EDC/NHS, gamma radiation or any combination thereof.

The bivalent or trivalent cation may be one or more of Ca²⁺, Ba²⁺, Mg²⁺, Sr²⁺, Al³⁺ and Fe³⁺.

According to a fourth aspect there is provided a shaped material comprising cross-linked alginate, wherein the cross-linked alginate originate from alginate from brown seaweed, and cellulose nanofibers, wherein the cellulose nanofibers originate from cellulose from the same brown seaweed. The cross-linked alginate may be obtained as discussed above.

The brown seaweed of the natural composition or shaped material may be selected from the group comprising Laminaria digitata, Laminaria hyperborean, Macrocystis pyrifera, Ascophyllum nodosum, Sargassum spp., Laminaria japonica, Ecklonia maxima and Lessonia nigrescens.

Brown seaweed species like Laminaria digitata, Laminaria hyperborean, Macrocystis pyrifera, Ascophyllum nodosum are mainly used for commercial alginate production, while species like Sargassum spp., Laminaria japonica, Ecklonia maxima and Lessonia nigrescens may be used when other brown seaweeds are not available because their alginate yield usually is low and weak (Khalil et al., 2018). As all these brown seaweeds contain alginate as well cellulose they may be suitable candidates for this kind of method. Depending on the brown seaweed species, the season, the growth site etc, the quality of the natural composition and shaped material may vary as the amount of cellulose and alginate may vary.

The concentration of cellulose in the natural composition or shaped material may be 10-40 wt % and the concentration of alginate may be 20-60 wt %.

As discussed above, depending on the brown seaweed species, the season, the growth site etc., the amount of cellulose and alginate may vary.

According to a fifth aspect there is provided a use of a natural composition described above in manufacturing of a shaped material.

Such a use may comprise the use of a 3D printer, wherein the natural composition is used as the ink.

The shaped material may be selected from a wire, a cord, a tube, a mesh, a bead, a sheet, a web, a disc, a cylinder, a coating, an interlayer, or an impregnate.

### BRIEF DESCRIPTION OF THE DRAWINGS

FIGS. 1a and 1b show SEM images of the cell wall structure of the raw materials, stripe and blade, respectively (scale bar: 100 μm). In FIGS. 1c and 1d are photographs of the raw materials and in FIGS. 1e and 1f photographs of the bleached structures. In FIGS. 1g and 1h 0optical microscopy (OM) images (above) and polarized optical microscopy (POM) images (below) at different fibrillation processing time (scale bar: 200 μm) are shown. Measured size distribution of the obtained nanofibers (scale bar: 600 nm) are shown in FIGS. 1i and 1j.

FIG. 2 shows rheological data for the inks, S-A-CNF and B-A-CNF, respectively. In FIG. 2a is shown flow curves, in FIGS. 2b and 2c are photographs of the ink gels at 2 wt. %. In FIG. 2d is shown the storage modulus G′, and in FIG. 2e the loss modulus G″ measured over time where a CaCl₂ solution was added 50 s after the measurement was started.

FIG. 3. shows compression evaluation of 3D printed S-A-CNF and B-A-CNF to determine their mechanical properties after crosslinking. In FIG. 3a compressive stress-strain curves up to 60% strain are shown. In FIG. 3b is shown photographs of the hydrogels after crosslinking. In FIG. 3c is shown the compressive stress and in FIG. 3d the compressive modulus at 30 and 60% strain.

### DETAILED DESCRIPTION

In the following is described a method for producing a composition and matrix comprising alginate and nanofibrillated cellulose originating from alginate and cellulose from the same brown seaweed species Laminaria digitate sample(s). The thus produced composition and matrix are evaluated and compared to reference material comprising nanofibrillated cellulose from other cellulose sources. It is to be understood that the methods steps and chemicals presented below are mere examples and should not be construed as limiting for the methods and composition/matrix. It is shown that the composition/matrix obtained from Laminaria digitate has characteristics similar to alginate/CNF compositions known in the art.

### Experimental Section

Materials. Brown seaweed, (Laminaria digitata) was provided by The Northern Company Co. (Træna, Norway) and used as a raw material. The fast-growing seaweed was cultivated in the North Atlantic Ocean on the Norwegian west coast and harvested in May 2017. Laminaria digitata consists of a holdfast (root-like), stipe (stem-like) and blade (leaf-like) structure (Misurcova et al., 2012). Its carbohydrate composition vary with season, geographic location, and age (Manns et al., 2017), as well as between the different parts of the seaweed (stipe and blade) (Black et al., 1950). Fresh samples were stored in wet condition in closed bags in a freezer before use. The stipe and blade of the seaweed were prepared in separate batches for comparison and utilization of the entire structure. Both materials, stipe and blade, were purified and nanofibrillated using equivalent processing conditions.

The chemicals used in the purification process, sodium hydroxide (NaOH), sodium chlorite (NaClO₂), acetic acid (CH₃COOH)), chemical composition (sodium bromide (NaBr)), and ionic crosslinking (calcium chloride (CaCl₂.2H₂O)) of laboratory grade were purchased from Sigma-Aldrich (Stockholm, Sweden) and were used as received. Deionized water was used for all experiments.

Preparation. The stipe and blade of the seaweed were left in room temperature for about 24 h in order to defrost and thereafter cut into smaller pieces, here about 1-3 cm², prior to purification using bleaching with NaClO₂ (1.7%) in an acetate buffer (pH 4.5) 80° C. for 2 h. In the purification process all colour was removed and the material was thereafter washed until a neutral pH was reached. The solid recovery was calculated as yields according to the following equation:

Yield (%)=W₁/W₀×100 (1),

where W₁ indicates the dry weight of the sample after the bleaching and W₀ indicates the initial dry weight of the seaweed. The presented yield is based on the average of three different batches.

The materials were nanofibrillated using an MKZA6-3 Supermasscolloider ultrafine friction grinder (Masuko Sangyo Co. Japan) with coarse silica carbide (SiC) grinding stones, and at a concentration of 2 wt. %. The nanofibrillation was operated in contact mode with a gap of the two disks set to −90 μm, at 1500 rpm. The total processing was 40 min and 30 min for the stipe and blade material, respectively. The prepared inks were denoted S-A-CNF (stipe) and B-A-CNF (blade).

The energy consumption for the fibrillation process was established by direct measurement of power using a power meter, Carlo Gavazzi, EM24 DIN (Italy) and the processing time. The energy demand was calculated from the product of power and time and the energy consumption for the fibrillation process is expressed as kWh per dry weight kg of the nanofibers. Samples were collected at regular intervals to assess the degree of fibrillation. The process was finalized when a plateau in viscosity was reached and no larger structures could be observed by microscope. The prepared inks were kept in a refrigerator at 6° C. prior to 3D printing of the hydrogels.

3D printing of biomimetic hydrogels. Cylindrical disks of S-A-CNF and B-A-CNF were 3D printed using the INKREDIBLE 3D bioprinter, CELLINK AB (Gothenburg, Sweden); a pneumatic-based extrusion bioprinter. The solid discs (10 mm diameter, 4 mm high, 6 layers) were designed in the CAD software 123D Design (Autodesk) and the created STL files were subsequently converted into g-code using Repetier-Host (Repetier Server) software. A nozzle diameter of 0.5 mm was used at a pressure of 5 kPa and dosing distance of 0.05 mm. The two ink formulations were 3D printed directly onto a glass petri dish and crosslinked thereafter in a bath of a 90 mM aqueous solution of CaCl₂ for 30 min directly on the petri dish and finally washed with deionized water. The printability was evaluated with concern to printer parameters and shape fidelity.

Chemical composition. The composition of the bleached stipe and the blade were assessed in terms of alginate and cellulose content; starting with a dry weight of 10 g. For the isolation of alginate, the procedure of Zubia et al., 2008 was followed using a formaldehyde alkali treatment method. The precipitate was washed with absolute ethanol followed by acetone, prior to drying for 24 h at 40° C. The alginate fraction was expressed as a percentage of dry weight.

The cellulose content was extracted following the method described by Siddhanta et al., 2009. In brief, the samples were defatted repeatedly with MeOH, followed by 600 ml NaOH (0.5M) solution at 60° C. overnight, washed and dried in room temperature. For removal of any remaining minerals, the dried material was re-suspended in a 200 ml solution of hydrochloric acid (5% v/v), washed and dried for 24 h at 40° C. The cellulose fraction was denoted as a percentage on a dry weight basis.

Polarized Optical Microscopy (POM). A polarizing microscope, Nikon Eclipse LV100N POL (Japan) and the imaging software NIS-Elements D 4.30 was used to assess the nanofibrillation process. Reference images without polarization filter were also captured. Viscosity. Viscosity measurements were also performed during the nanofibrillation using a Vibro Viscometer SV-10, (A&D Company, Ltd, Japan), at a constant shear rate. The velocity (shear rate) of the sensor plates keeps periodically circulating from zero to peak because sine-wave vibration is utilized, at a frequency of 30 Hz. The viscosity measurements were repeated once the temperature of the samples had been stabilized to 22.3±1.0° C. to confirm that a plateau in viscosity had been reached during fibrillation. The presented values are an average of three measurements for each sample.

Atomic Force Microscopy (AFM). The morphology was studied after the nanofibrillation using an Atomic Force Microscopy (AFM). The fibrillated sample suspension (0.01 wt-%) was dispersed and deposited by spin coating onto a clean mica for imaging. The measurements were performed on a Veeco Multimode Scanning Probe, USA in tapping mode, with a tip model TESPA (antimony (n) doped Si), Bruker, USA. The nanofiber size (width) was measured from the height images using the Nanoscope V software and the average values and deviations presented are based on 50 different measurements. All measurements were conducted in air at room temperature.

Scanning Electron Microscopy (SEM). The cross-sections of the stipe and blade were observed using a using a SEM JCM-6000 NeoScope (JEOL, Tokyo, Japan) at an acceleration voltage of 15 kV to study their cell wall structures. In addition, the cross-section of the nanofilms were observed. All samples were coated using a coating system machine (Leica EM ACE200, Austria) with a platinum target. The coating was performed within a vacuum of approximately 6×10⁻⁵ mbar, under a current of 100 mA, for 20 s to obtain a coating thickness of 25 nm.

Rheology. The rheological behaviour of the hybrid-inks, S-A-CNF and B-A-CNF were analysed using the Discovery HR-2 rheometer (TA Instruments, UK) at 25° C. A cone-plate (20 mm) was used and the shear viscosity was measured at shear rates from 0.01-1000 s⁻¹. Furthermore, the change in moduli while cross-linking the ink was measured with a plate-plate configuration (8 mm, gap 500 μm). The oscillation frequency measurements were conducted at 0.1% strain, based on oscillation amplitude sweeps to establish the LVR, and at a frequency of 1 Hz for 10 min. 50 s after the measuring was started, a 1 mL drop of 90 mM CaCl₂ solution was added around the inks causing gelling while simultaneously measuring the storage and loss modulus.

Compression properties. Uniaxial unconfined compression tests of the 3D printed and cross-linked hydrogels were carried out using a dynamic mechanical analyser DMA Q800 (TA Instruments, New Castle, USA) at 25° C. The hydrogels were preloaded using a load of 0.05 N, and subsequently compressed up to a strain of 100%, and at a strain rate of 10% min⁻¹. The materials were compared by the stress and tangent modulus at 30% and 60% compressive strain level, respectively. The disks with dimensions of 10 mm in diameter and a height of 4 mm were tested 6 times for each material; the average results are reported.

### Results and Discussion

Purification and characterization of raw material. The yield and chemical composition after the pretreatment of the raw materials is presented in Table 1.

TABLE 1
Yield calculation, and cellulose and
alginate content after purification

| | Initial | Weight | Total | | |
|---|---|---|---|---|---|
| Raw | weight | after bleaching | yield | Cellulose | Alginate |
| Materials | [g] | [g] | [%] | [wt. %] | [wt. %] |
| Stipe | 70 | 49.7 | 71 ± 8 | 33 ± 6 | 45 ± 13 |
| Blade | 70 | 51.8 | 74.2 ± 7 | 23 ± 3 | 46 ± 11 |

The objective of the purification of the seaweed was to remove the colour pigments and other impurities, while maintaining as much of the inherently high alginate content found in brown seaweed, together with the cellulose content. Indeed, the yield of the stipe and blade were as high as 71% and 74%, respectively after the bleaching procedure (Table 1). These values can be compared to that of wood after direct bleaching, namely about 70%, yet mainly composed of hollocellulose.

An alginate content of 25-30 wt. % and cellulose content of 10-15 wt. % have previously been reported for the raw seaweed, Laminaria Digitata harvested in Scotland during May (Schiener et al., 2015). From Table 1, after bleaching, the alginate and cellulose contents were higher, yet their relative percentage to each other was maintained. The stipe measured a higher cellulose content, though the significance is questionable considering the standard deviations, which might reflect the heterogeneity of the raw material even within a specie (Manns et al., 2014). There are only a limited number of studies that have measured the compositional content of the different parts of brown seaweed, and for Laminaria Digitata, a cellulose content of 6-8 wt. % and 3-5 wt. % have been reported for the stipe and the blade, respectively (Black et al., 1950). However, the cellulose content is highly dependent on several factors such as: measuring methods, geographical, seasonal, and age to mention a few (Schiener et al., 2015).

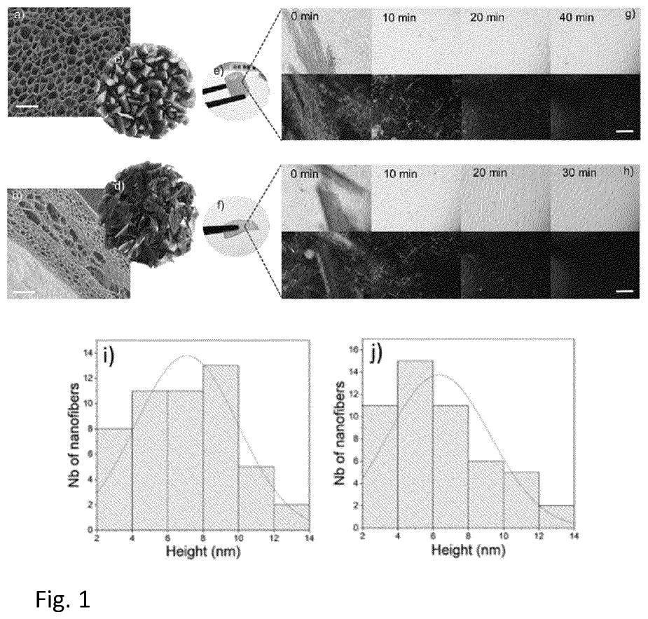

Nanofibrillation process and characterization of inks. The nanofibrillation of the purified materials was carried out using viscosity measurements and POM/OM to assess the degree of fibrillation throughout the process. The route from the raw materials to nanoscale is shown in FIG. 1.

The viscosity may be used as an indication of the degree of fibrillation, where the viscosity plateau has signified a strong network formation of separated nanofibers with a maintained length (Berglund et al., 2016).

The increased viscosity and plateau of both S-A-CNF and B-A-CNF were clearly observed from the samples measured in room temperature, namely 3289, and 2102 mPas, respectively. When comparing these viscosity values to that of wood pulp, the viscosity plateau at 1565 mPa s was significantly lower and reached first after 90 min of fibrillation.

In FIGS. 1c and 1d photographs of the different parts of brown seaweed, stipe and blade, are shown. From the cross-sectional views, FIGS. 1a and 1b, differences of the cell wall structures of the different parts of brown seaweed, stipe and blade, are apparent. A more organized structure was observed for the stipe (FIGS. 1a, 1c), compared to the more layer-like structure of the blade (FIGS. 1b, 1d), displaying a wide range of pore-sizes. Completely white structures were obtained after the bleaching process (FIGS. 1e, 1f). In FIGS. 1g and 1h optical microscopy (OM) images (above) and polarized optical microscopy (POM) images (below) at different fibrillation processing time (scale bar: 200 μm) are shown. The nanofibrillation of the stipereached a maximum viscosity at an energy demand of 1.5 kWh/kg. In comparison, the blade had a slightly lower energy demand throughout the process, and the maximum viscosity was reached at an energy demand of 1.0 kWh/kg. The slightly higher energy demand of the stipe could be explained by its higher cellulose content (Table 1), which might acquire more energy to be separated. In addition, the arrangement of cellulose and alginate in the stipe appear to be more consolidated in thicker cell walls as seen in FIG. 1a). The nanofibers of S-A-CNF and B-A-CNF were in average 7±3 and 6±3 nm, respectively. Measured size distribution of the obtained nanofibers (scale bar: 600 nm) are shown in FIGS. 1i and 1j.

The measured energy consumption was, remarkably low for the nanofibrillation of both seaweed structures, in comparison to that of commercially bleached wood karft pulp, that reached a maximum viscosity at 8.4 kWh/kg under the similar processing conditions (Berglund et al., 2017). The importance of hemicellulose present for the process efficiency of nanofibrillation of wood pulp have previously been reported using ultrafine grinding (Iwamoto et al., 2008). The low energy demand suggest that the presence of alginate during nanofibrillation may act beneficial for the separation of nanofibers.

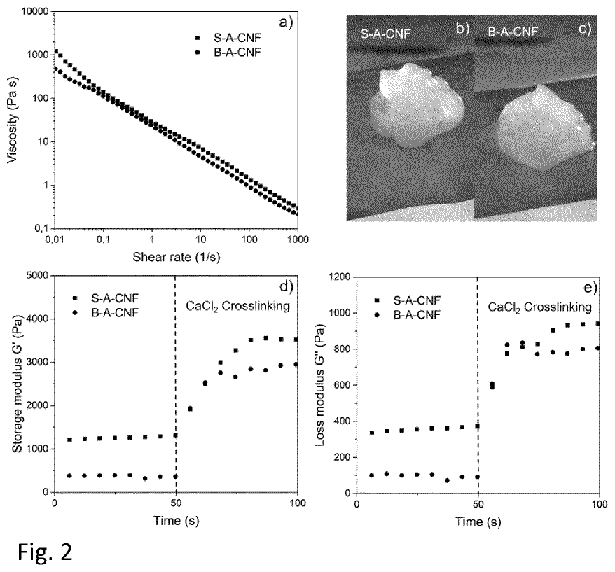

3D printability and characterization of biomimetic hydrogels. The rheological behaviour of the inks were studied to evaluate their suitability for 3D printing. In FIG. 2a a shear-thinning behaviour is observed for both S-A-CNF and B-A-CNF inks, similar to viscosity curves previously reported for commercial alginate mixed with CNF (Abouzeid et al., 2018), as well as pure CNF (Markstedt et al., 2015). For S-A-CNF, the initial viscosity was 1224 Pa s and it decreased to 0.3 Pa s upon increasing the shear rate to 1000 1/s, in comparison to B-A-CNF which initially was lower at 578 Pa s, and dropped to 0.2 Pa s at a shear rate of 1000 1/s. Also, the higher viscosity of S-A-CNF compared to B-A-CNF can be visually seen in FIG. 2b and FIG. 2c. The high viscosity at low shear rates and the shear thinning behaviour with increasing shear rates provide shape fidelity during printing. To maintain the structural integrity after printing, crosslinking of the alginate is required, however. Hence, the gelling behaviour of the inks was studied by measuring the loss—(G′) and storage (G″) modulus as a function of time while crosslinking with CaCl₂ (see FIGS. 2d and 2e). Both the storage modulus, FIG. 2d, and loss modulus, FIG. 2e, displayed an instant increase upon addition of CaCl₂ solution at 50 s, and become gradually linear after additionally 50 s. The time was measured for additionally 5 min to confirm this plateau. The higher storage modulus of S-A-CNF reflects a higher degree of cross-linking, in turn resulting in a higher strength or mechanical rigidity.

3D-printability and crosslinking enables the use of inks in a wide range of applications that for example requires specific shapes for wound dressing (Leppiniemi et al., 2017), or even 3D-printing of living tissues and organs (Markstedt et al., 2015). The printability and stability of 3D discs from S-A-CNF and B-A-CNF inks, as prepared at 2 wt. % solid content, were studied and the printing parameters were tuned through a trial-and-error method. Both inks could be printed without collapse of the structure, yet S-A-CNF displayed a better shape fidelity likely attributed to the higher viscosity.

A minor shrinkage of the diameter and some swelling in the centre, appearing as a slightly convex surface were observed after crosslinking of the discs. These tendencies of shape deformation after CaCl₂ crosslinking have previously been reported for 3D printed alginate/CNF hydrogels (Markstedt et al., 2015; Leppiniemi et al., 2017). The behaviour might reflect inadequate homogeneity of the diffusion based CaCl₂ crosslinking approach.

The ionic crosslinking of alginate using CaCl₂ has been widely studied and by varying parameters such as crosslinking ratio (Freeman et al., 2017), and crosslinking time (Giuseppe et al., 2018) the mechanical properties of printed hydrogels can be tuned. However, other factor such as: molecular weight and M/G ratio, originating from the raw material and its alginate extraction process have a high influence both on crosslinking behaviour and fundamental mechanical behaviour.

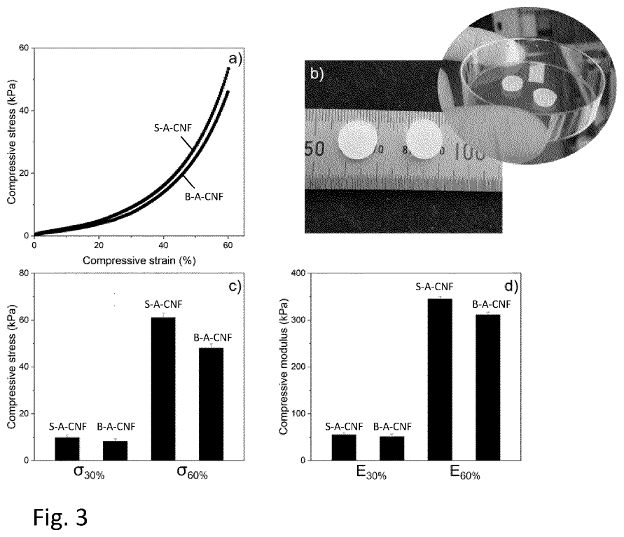

The 3D printed S-A-CNF and B-A-CNF hydrogels were evaluated in compression to determine their mechanical properties after crosslinking, as presented in FIG. 3.

Since the compressive stress and strain curves revealed a viscoelastic non-linear stress-strain behaviour, the compressive modulus and stress at 30 and 60% strain were used for mechanical characterization (FIG. 3a) of the 3D printed hydrogels (see FIG. 3b).

In FIG. 3c and FIG. 3d, it is shown that S-A-CNF has an overall higher compressive property in comparison to B-A-CNF. This is in good agreement with the rheological behaviour and could be explained by a higher amount of CNF, reinforcing the structure.

However, the stiffness of alginate hydrogels is directly related to its crosslinking, and still the S-A-CNF with a lower amount of alginate displays a higher stiffness as seen in FIG. 3d.

In Laminara digitata, a higher amount of alginate rich in guluronic acid (G) were shown for the stipe when compared to the blade of the seaweed (Peteiro et al. 2018), thus equivalent with a lower M/G ratio in the stipe. Alginates with lower M/G ratio are known to display a higher affinity towards crosslinking (mechanical rigidity), and the gel strength of alginate is mainly dependent on content and length of the guluronic acid. A lower M/G ratio of the alginate in the S-A-CNF hydrogel, compared to that of B-A-CNF may further contribute to the higher compressive properties.

Notable is also that the maximum compressive stress could be measured at around 80% strain for the B-A-CNF hydrogel (175.2 kPa±3). At this strain the B-A-CNF hydrogel fractured, while the S-A-CNF hydrogel was compressed without any visual fractures. The combination of the alginate of S-A-CNF ink with its CNF content appear to assemble into a biomimetic hydrogel with high compressive stiffness and strength, yet highly flexible.

The above described composition may be used in bioprinting with living cells for example as bioinks in 3D bioprinting of soft-tissue.

Crucial for obtaining the properties of the composition discussed above, i.e. the rheological behaviour and in turn the printability of the composition, is the extraction process of both alginate and cellulose nanofibers. For example, alginate extraction-purification from brown seaweed using three different routes was shown by Gomez et al (2009) to result in significant differences in rheological and gelation behaviour. Another example by Hiasa et al (2016) demonstrated the difference between pectin-containing cellulose nanofibers (based on the natural raw material structure) opposed to the addition of commercial pectin to cellulose nanofibers. The commercial pectin that was added did not interact with the purified cellulose nanofibers, thus significantly limiting the dispersion properties (and, hence, printability) compared to the natural pectin-containing nanofibers. Hence, to obtain the printable composition described above, the alginate and cellulose nanofibers should originate from the same brown seaweed sample(s) and, hence, have a natural composition of alginate and cellulose.

### REFERENCES

Abouzeid, R. E.; Khiari, R.; Beneventi, D.; Dufresne, A. Biomimetic mineralization of three-dimensional printed alginate/TEMPO-oxidized cellulose nanofibril scaffolds for bone tissue engineering. Biomacromolecules 2018 19 (11), 4442-4452.

Berglund, L.; Anugwom, I.; Hedenström, M.; Aitomäki, Y.; Mikkola, J. P.; Oksman, K. Switchable ionic liquids enable efficient nanofibrillation of wood pulp. Cellulose 2017 24, 3265-3279.

Berglund, L.; Noël, M.; Aitomäki, Y.; Öman, T.; Oksman, K. Production potential of cellulose nanofibers from industrial residues: efficiency and nanofiber characteristics. Ind Crop Prod 2016 92, 84-92.

Black, W. A. P. The seasonal variation in the cellulose content of the common Scottish Laminariaceae and Fucaceae. J. Marine Biological Association of the United Kingdom 1950, 29 (2) 379-387.

Chinga-Carrasco, G. Potential and limitations of nanocelluloses as components in biocomposite inks for three-dimensional bioprinting and for biomedical devices. Biomacromolecules 2018 19 (3), 701-711.

Di Giuseppe, M.; Law, N.; Webb, B.; Macrae, R. A.; Liew, L. J.; Sercombe, T. B.; Dilley, R. J.; Doyle, B. J. Mechanical behaviour of alginate-gelatin hydrogels for 3D bioprinting. J. Mech Behav Biomed Mater 2018 79, 150-157.

Falsini, S.; Bardi, U.; Abou-Hassan, A.; Ristori, S. Sustainable strategies for large-scale nanotechnology manufacturing in the biomedical field. Green Chem 2018, 20, 3897-3907.

Freeman, F. E.; Kelly, D. J. Tuning alginate bioink stiffness and composition for controlled growth factor delivery and to spatially direct MSC fate within bioprinted tissues. Scientific Reports 2017 7(17042), 1-12.

Gomez, C. G.; Lambrecht, V. P.; Lozano, J. E.; Rinaudo M.; Villar, M. A. Influence of the extraction-purification conditions on final properties of aliginates obtained from brown algae (Macrocystis pyrifera). International Journal of Biological Macromoleucles 2009, 44, 365-371.

Hiasa, S.; Kumagai, A., Endo; T., Edashige, Y. Prevention of Aggregation of Pectin-Containing Cellulose Nanofibers Prepared from Mandarin Pee. Journal of Fiber Science and Technology 2016, 72(1), 17-26.

Iwamoto, S.; Abe, K.; Yano, H. The effect of hemicelluloses on wood pulp nanofibrillation and nanofiber network characteristics. Biomacromolecules 2008 9 (3), 1022-1026.

Khalil, HPSA.; Lai, T K.; Tye, Y Y.; Rizal, S.; Chong, E W N.; Yap, S W.; Hamzah, A A.; Fazita, M R N.; Paridah, M T. A review of extractions of seaweed hydrocolloids: Properties and applications', Express Polymer Letters, 2018 12(4), 296-317.

Kong, H. J.; Lee, K. Y.; Mooney, D. J.; Decoupling the dependence of rheological/mechanical properties of hydrogels from solids concentration. Polymer 2002, 43 (23), 6239-6246.

Leppiniemi, J.; Lahtinen, P.; Paajanen, A.; Mahlberg, R.; Metsä-Kortelainen, S.; Pinomaa, T.; Pajari, H.; Vikholm-Lundin, I.; Pursula, P.; Hytönen, V. P. 3D-printable bioactivated nanocellulose-alginate hydrogels. ACS Appl Mater Interfaces 2017 9 (26), 21959-21970.

Liling, G.; Di, Z.; Jiachao, X.; Xin, G.; Xiaoting, F.; Qing, Z. Effects of ionic crosslinking on physical and mechanical properties of alginate mulching films. Carbohydr Polym 2016 136, 259-265.

Manns, D.; Deutschle, A. L.; Saake, B.; Meyer, A. S. Methodology for quantitative determination of the carbohydrate composition of brown seaweeds (Laminariaceae). RSC Adv 2014 4, 25736- 25746.

Manns, D.; Nielsen, M. M.; Bruhn, A.; Saake, B.; Meyer, A. S. Compositional variations of brown seaweeds Laminaria digitata and Saccharina latissima in Danish waters. J. Appl Phycol 2017, 29 (3), 1493-1506.

Markstedt, K.; Mantas, A.; Tournier, I.; Martinez Ávila, H.; Hägg, D.; Gatenholm, P. 3D bioprinting human chondrocytes with nanocellulose-alginate bioink for cartilage tissue engineering applications. Biomacromolecules 2015, 16, 1489-1496.

McHugh D. J.: A guide to seaweed industry. FAO Fisheries and Aquaculture Department, Rome (2003).

Misurcova, L. In handbook of marine macroalgae: biotechnology and applied phycology, 1st ed.; Se-Kwon, K., Ed.; JohnWiley & Sons, Ltd.; New Delhi, India, 2012; p 181-182.

Naseri, N.; Deepa, B.; Mathew, A. P.; Oksman, K.; Girandon, L. Nanocellulose-based interpenetrating polymer network (IPN) hydrogels for cartilage applications. Biomacromolecules 2016 17 (11), 3714-3723.

Peteiro, C. In alginates and their biomedical applications, Rehm, B.; Moradali, M. Eds.; Springer Series in Biomaterials Science and Engineering vol 11; Springer, Singapore, 2017; p 27-58.

Schiener, P.; Black, K. D.; Stanley, M. S.; Green, D. H. The seasonal variation in the chemical composition of the kelp species Laminaria digitata, Laminaria hyperborea, Saccharina latissima and Alaria esculenta. J. Appl Phycol 2015 27, 363-373.

Siddhanta, A. K.; Prasad, K.; Meena, R.; Prasad, G.; Mehta, G. K.; Chhatbar, M. U.; Oza, M. D.; Kumar, S.; Sanandiya, N. Profiling of cellulose content in Indian seaweed species. Bioresour Technol 2009 100, 6669-6673.

Siqueira, P.; Siqueira, É.; de Lima, A. E.; Siqueira, G.; Pinzon-Garcia, A. D.; Lopes, A. P.; Cortés Segura, M. E.; Isaac, A.; Vargas Pereira, F.; Botaro, V. R. Three-dimensional stable alginate-nanocellulose gels for biomedical applications: towards tunable mechanical properties and cell growing. Nanomaterials 2019 9 (1), 78-100.

Zubia, M.; Payri, C.; Deslandes, E. Alginate, mannitol, phenolic compounds and biological activities of two range-extending brown algae, Sargassum mangarevense and Turbinaria ornate (Phaeophyta: Fucales), from Tahiti (French Polynesia). J. Appl Phycol 2008 20, 1033-1043.

1. A natural composition for 3D printing comprising:
alginate from brown seaweed, and
cellulose nanofibers,
wherein the cellulose nanofibers originate from cellulose from the same brown seaweed sample(s) as the alginate.

2. The natural composition of claim 1, wherein a solid content of the natural composition is 2-10 wt %.

3. A method for preparing a natural composition comprising alginate and cellulose nanofibers, wherein the method comprises the steps of:
providing a material of brown seaweed,
purifying the material to remove impurities from the brown seaweed comprising the alginate and cellulose, and
nanofibrillating the cellulose of the purified material.

4. The method of claim 3, wherein the step of purifying the material comprises the use of one or more cellulose bleaching substances.

5. A method for preparing a shaped material comprising alginate and cellulose nanofibers, the method comprising the method of claim 3, and further steps of:
forming a shaped material of the composition, and
crosslinking the alginate.

6. The method of claim 5, wherein the step of crosslinking the alginate comprises the use of a bivalent or trivalent cation, a peroxide, a vinylsilane, UV light, EDC/NHS, gamma radiation or any combination thereof.

7. The method of claim 6, wherein the bivalent or trivalent cation is one or more of Ca²⁺, Ba²⁺, Mg²⁺, Sr²⁺, Al³⁺ or Fe³⁺.

8. A shaped material comprising the composition of claim 1, wherein the alginate is cross-linked.

9. The natural composition of claim 1, wherein the brown seaweed is selected from the group consisting of Laminaria digitata, Laminaria hyperborean, Macrocystis pyrifera, Ascophyllum nodosum, Sargassum spp., Laminaria japonica, Ecklonia maxima and Lessonia nigrescens.

10. The natural composition of claim 1, wherein the concentration of cellulose is 10-40 wt % and the concentration of alginate is 20-60 wt %.

11. (canceled)

12. (canceled)

13. The method of claim 3, wherein the brown seaweed is selected from the group consisting of Laminaria digitata, Laminaria hyperborean, Macrocystis pyrifera, Ascophyllum nodosum, Sargassum spp., Laminaria japonica, Ecklonia maxima and Lessonia nigrescens.

14. The shaped material of claim 8, wherein the brown seaweed is selected from the group consisting of Laminaria digitata, Laminaria hyperborean, Macrocystis pyrifera, Ascophyllum nodosum, Sargassum spp., Laminaria japonica, Ecklonia maxima and Lessonia nigrescens.
